(19) United States
(12) Patent Application Publication
(10) Pub. No.: US 2024/0268959 A1
(43) Pub. Date: Aug. 15, 2024

(54) DEVICES AND METHODS FOR FORMING GUIDED ANGIOGENESIS AND ENHANCED VASCULARIZATION IN HEART TISSUE

(71) Applicant: Helios Cardio Inc., Weston, MA (US)

(72) Inventors: Yiannis Monovoukas, Weston, MA (US); Kevin Cornwell, Holliston, MA (US); Peter Jackson, Wellesley, MA (US)

(73) Assignee: Helios Cardio Inc., Weston, MA (US)

(21) Appl. No.: 18/433,595

(22) Filed: Feb. 6, 2024

Related U.S. Application Data
(60) Provisional application No. 63/484,938, filed on Feb. 14, 2023.

(51) Int. Cl. A61F 2/24; A61F 2/00; A61L 27/36; A61L 27/38; A61L 27/54; A61L 27/56

(52) U.S. Cl. CPC A61F 2/2481; A61L 27/3633; A61L 27/3679; A61L 27/3834; A61L 27/54; A61L 27/56; A61F 2002/0081; A61F 2002/0086; A61F 2240/001; A61L 2430/20

(57) ABSTRACT

Devices and methods for forming guided angiogenesis and enhanced vascularization in ischemic heart tissue are described. In some embodiments, a biopolymer scaffold material may comprise a plurality of through holes (e.g., slits, cutouts, and/or other appropriate types of through holes) that may guide angiogenesis and promote enhanced revascularization in ischemic heart tissue. In some embodiments, microneedles may be used to form a plurality of microchannels in ischemic heart tissue. The plurality of microchannels may be patterned to form guided vascularization such that vascularization may be directed from non-ischemic heart tissue to the ischemic heart tissue. In some embodiments, a biopolymer scaffold material including through holes formed therein applied to ischemic heart tissue may be combined with a plurality of microchannels formed in the ischemic heart tissue to improve microvascularization of the ischemic heart.

### RELATED APPLICATIONS

This application claims the benefit of priority under U.S.C. § 119(e) of U.S. Provisional Application Ser. No. 63/484,938, filed Feb. 14, 2023, the disclosure of which is incorporated herein by reference in its entirety.

### BACKGROUND

The leading cause of morbidity and mortality in the US and globally is ischemic heart disease that causes chest pain, shortness of breath, and fatigue and can lead to heart attack and potentially cardiac arrest. Ischemic heart disease is typically caused by obstruction of the coronary arteries feeding the heart leading to myocardial infarction (MI). An additional subset of patients have clinical symptoms of ischemic heart disease but without clear obstructions of the larger coronary arteries. These patients have coronary microvascular disease (CMD) caused by disorders of the microcirculation. Primary treatments for increasing perfusion of the ischemic myocardium includes drugs and/or mechanical reperfusion of the heart through stenting of blocked vessels and/or placement of coronary artery bypass grafts. MI or progressive heart failure could be limited, prevented, or reversed through increasing microvasculature of ischemic regions of the heart.

### SUMMARY

In some embodiments, an ischemic heart repair device is provided. The ischemic heart repair device may comprise a biopolymer scaffold material which includes a plurality of interconnected pores. The biopolymer scaffold material may have a thickness that is greater than or equal to approximately 0.2 mm and less than or equal to approximately 6 mm. The biopolymer scaffold material may also include a plurality of through holes formed therein that extend from a first surface of the biopolymer scaffold material to a second opposing surface of the biopolymer scaffold material. The plurality of through holes may have an average maximum transverse dimension that is less than or equal to 1 mm.

In some embodiments, a method of forming guided vascularization in heart tissue is provided. The method may comprise applying a biopolymer scaffold material having a plurality of interconnected pores to an epicardium of an ischemic portion of a heart of a subject. The biopolymer scaffold material may have a thickness that is greater than or equal to approximately 0.2 mm and less than or equal to approximately 6 mm. The biopolymer scaffold material may have a plurality of through holes formed therein that extend from a first surface of the biopolymer scaffold material to a second opposing surface of the biopolymer scaffold material. The plurality of through holes may have an average maximum transverse dimension that is less than or equal to 1 mm.

In some embodiments, a method of promoting vascularization in heart tissue is provided. The method may comprise forming a plurality of microchannels in an ischemic portion of a subject's heart. The plurality of microchannels may extend through an epicardium and partially through a myocardium of the subject's heart.

In some embodiments, a method of promoting vascularization in heart tissue is provided. The method may comprise forming a plurality of microchannels in a subject's heart in a region including a healthy portion of the subject's heart and an ischemic portion of the subject's heart. In some embodiments, at least a portion of the plurality of microchannels are interconnected.

In some embodiments, a device for promoting guided and enhanced vascularization in heart tissue is provided. The device may comprise at least one microneedle configured to be moved between a retracted configuration and an extended configuration. The at least one microneedle may be configured to form at least one microchannel in the heart of a subject in the extended configuration. The at least one microneedle may have an average maximum transverse dimension of less than or equal to 1 mm. The at least one microneedle may also have an average length in the extended configuration of greater than or equal to 5 mm and less than or equal to 15 mm.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic of a heart with an ischemic portion, according to an embodiment;

FIG. 2A is a schematic of a biopolymer scaffold material comprising a plurality of through holes, according to one embodiment;

FIG. 2B is a schematic of a biopolymer scaffold material comprising a plurality of through holes, according to another embodiment;

FIG. 3A is a schematic of a biopolymer scaffold material and a plurality of microneedles located adjacent to an ischemic portion of a heart, according to one embodiment;

FIG. 3B is a schematic of a biopolymer scaffold material located adjacent to an ischemic portion of a heart, and a plurality of microchannels formed in the ischemic portion of the heart, according to one embodiment;

FIG. 4A is a schematic of a biopolymer scaffold material and a plurality of microneedles interacting with an ischemic portion of a heart, according to another embodiment;

FIG. 4B is a schematic of the heart of FIG. 4A after a plurality of microchannels are formed in the ischemic portion of the heart, according to another embodiment;

FIG. 5A is a schematic of a biopolymer scaffold material and a plurality of microneedles located interacting with an ischemic portion of a heart, according to another embodiment;

FIG. 5B is a schematic of the heart of FIG. 5A after a plurality of straight microchannels are formed in the ischemic portion of the heart, according to another embodiment;

FIG. 5C is a schematic of the heart of FIG. 5B and a plurality of angled microneedles, according to another embodiment;

FIG. 5D is a schematic of the heart of FIG. 5C after a plurality of angled microchannels interconnecting the separately formed microchannels have been formed in the ischemic portion of the heart, according to another embodiment;

FIG. 6A is a schematic of a micro needling device, according to one embodiment;

FIG. 6B is a schematic of a micro needling device, according to another embodiment;

FIG. 6C is a schematic of a micro needling device, according to another embodiment;

FIG. 6D is a schematic of a micro needling device, according to another embodiment;

FIG. 6E is a schematic of a micro needling device, according to another embodiment;

FIG. 6F is a schematic of a micro needling device, according to another embodiment;

FIG. 7 is a schematic of a biopolymer scaffold material and a micro needling device, according to an embodiment;

FIG. 8A is a schematic of a heart with a biopolymer scaffold material applied to an ischemic portion of the heart, according to one embodiment;

FIG. 8B is a schematic of a heart with a biopolymer scaffold material applied to an ischemic portion of the heart, according to another embodiment;

FIG. 9 is a schematic of a heart with a biopolymer scaffold material applied to an ischemic portion of the heart, according to another embodiment; and

FIG. 10 is a method forming vascularization in a portion of a heart, according to an embodiment.

### DETAILED DESCRIPTION

Following a heart attack (myocardial infarction or MI) or during progressive heart failure (HF), perfusion of the myocardium is significantly impaired. Primary treatments include drugs and the mechanical reperfusion of the heart commonly through stenting of blocked vessels and/or surgical placement of coronary artery bypass grafts. However, areas of the heart often remain poorly perfused which may result in continually remodeling of heart tissue into fibrous scar tissue which may further affect heart function as HF progresses. Additionally, some patients may suffer from coronary microvascular disease (MVD) where the larger vessels may remain open but the small capillaries are blocked or inhibited leading to heart dysfunction. Some patients may ultimately end up on a ventricular assist device (VAD) or receive heart transplants to fix the problems of the heart. Strategies to increase the microvasculature of damaged or poorly vascularized myocardium may limit, prevent, or reverse the progression of heart failure thereby delaying the need for VADs or heart transplants, and may even help prevent cardiac arrest.

In addition to the use of stents and coronary artery bypass grafts to treat heart conditions described above, technologies such as laser Transmyocardial Revascularization (TMR) have been employed to improve revascularization in ischemic heart tissue. TMR primarily functions by applying laser energy directly to target areas of the heart tissue, thereby forming a plurality of microchannels through the heart tissue. These microchannels may improve blood flow to ischemic regions of the heart by allowing for fresh blood to perfuse through the heart wall. Specifically, TMR has been predominantly used to treat angina in patients whose heart conditions have not been relieved by other traditional revascularization methods. While TMR has found benefit in reducing pain associated with angina of a patient, the inventors have recognized that traditional TMR devices have certain disadvantages. For example, TMR may damage portions of the heart tissue during the process of forming microchannels, and as a result may yield excessive blood loss from the region of the heart in which the microchannels are formed. In another example, TMR penetrates the heart tissue past the endocardium and into the ventricle during the formation of these microchannels. The inventors have also found that while TMR may provide benefits in reducing the severity of angina associated with a patient (i.e., as a result of denervation), that the effects of TMR in promoting revascularization in ischemic areas of the heart may be minimal.

In view of the above, the inventors have recognized that there is a need for devices and/or methods which improve revascularization in ischemic portions of heart tissue which may help to limit, prevent, and/or reverse a variety of heart conditions detailed above.

The inventors have also recognized the benefits associated with applying biopolymer scaffold materials to an ischemic portion of the heart to promote vascularization of the ischemic portion of the heart. In some embodiments, the biopolymer scaffold materials may include a plurality of interconnected pores and a plurality of through holes passing from one surface of a biopolymer scaffold material to a second opposing surface of the biopolymer scaffold material. When appropriately sized and distributed around an area of the biopolymer scaffold material, the through holes and overall structure of the biopolymer scaffold material may help to promote and guide the desired vascularization of the ischemic heart tissue that the biopolymer scaffold material is applied to. For example, a biopolymer scaffold material including a plurality of through holes formed therein may be applied to an epicardium of an ischemic portion of a heart to promote vascularization within the associated ischemic heart tissue as elaborated on further below.

As noted above, according to some embodiments, the inventors have further recognized that vascularization may be improved by forming a plurality of through holes with particular size ranges and distributions in the above-mentioned biopolymer scaffold materials. Specifically, such through holes may promote angiogenesis (i.e., the formation of new blood vessels) in an ischemic portion of a patient's heart tissue. In some embodiments, angiogenesis may be directed from healthier, perfused areas of the heart to areas of the heart with poor or limited perfusion by trapping blood components (e.g. platelets and/or clotting factors) within the decellularized extracellular matrix, thereby creating revascularization. In some such embodiments, the through holes may be actively filled during a surgical procedure with platelets, platelet gel, or platelet-rich plasma (PRP). The through holes may also be inactively filled via surgical bleeding in situ. By promoting revascularization in ischemic portions of the heart tissue, blood supply to ischemic areas may be increased and the generation and/or maintenance of heart tissue (e.g., myocardial tissue) may be supported.

In view of the above, a biopolymer scaffold material may be loaded with one or more populations of cells configured to facilitate or otherwise aid in vascularization of the biopolymer scaffold and/or the underlying heart tissue. For example, the biopolymer scaffold material may be loaded with one or more populations of cells prior to implantation. In another example, the biopolymer scaffold material may be loaded with one or more populations of cells while in vivo via cells transported to the biopolymer scaffold material in any of the ways noted above relative to the transport of blood components to the biopolymer scaffold material. In some embodiments, the biopolymer scaffold material may be loaded with one or more populations of cells both prior to implantation and while in vivo. It should be understood that any appropriate type of cell population may be used as the disclosure is not so limited including, for example, autologous cells and/or engineered cells. This may include in some embodiments, allogenic cells such as engineered stem cells. In either case, one or more populations of cells may be positioned adjacent to ischemic heart tissue where one or more populations of cells may synergistically aid in the formation of re-vascularized heart tissue in combination with the above noted biopolymer scaffold materials applied to the heart tissue.

To help facilitate the growth of vascularized tissue, in some embodiments, it may be desirable to provide an appropriate growth factor to stimulate growth of one or more cell populations and/or heart tissue in general. For example, a growth factor may be absorbed, coated, or otherwise loaded into a biopolymer scaffold material for subsequent release after implantation. Appropriate types of growth factors may include, but are not limited to platelet derived growth factor (PDGF), fibroblast growth factor (FGF), transforming growth factor (TGF), vascular endothelial growth factor (VEGF), and platelet-derived angiogenesis factor (PDAF). Without wishing to be bound by theory, the use of such growth factors on ischemic portions of the heart tissue may help facilitate revascularization and/or regeneration of the ischemic tissue.

The through holes in the biopolymer scaffold materials disclosed above may be provided in any suitable number and in any suitable arrangement to stimulate and guide the growth of vasculature. In some embodiments, a suitable number of through holes in the biopolymer scaffold material may be greater than or equal to 10, 50, 100, 200, 300, 400, 500 or greater. In some embodiments, the number of through holes may be less than or equal to 1000, 500, 400, or any other appropriate number. Combinations of foregoing are contemplated including, for example, a number of through holes that is between or equal to 10 and 1000, 100 and 1000, and/or any other appropriate combination. Numbers of through holes both greater than and less than those noted above are also contemplated as the disclosure is not so limited.

The through holes present in a biopolymer scaffold material may also be arranged to have any suitable area density such that the material still has sufficient structural integrity for use in vivo on a beating heart. In some embodiments, a suitable area density of the through holes as a percentage relative to the total area of the biopolymer scaffold material (e.g., an area of the through holes in a plane parallel to a surface of the material intended to be oriented towards a heart once implanted divided by a total area of that surface) may be greater than or equal to 5%, greater than or equal to 10%, greater than or equal to 15%, greater than or equal to 20%, greater than or equal to 25%, greater than or equal to 30%, greater than or equal to 35% greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, or any other appropriate percentage. Correspondingly, the percentage of the area of the through holes versus the total area of a surface of the biopolymer scaffold material intended to be oriented towards a heart during use in vivo may be less than or equal to 80%, 75%, 70%, 65%, 60%, 55%, 50%, and/or any other appropriate percentage. Combinations of foregoing are contemplated including, for example, a percentage that is between or equal to 5% and 80%. While any suitable area density of through holes may be used as noted above, the inventors have found that in a preferred embodiment the area density of through holes may be approximately 20% (e.g., 15% to 25%) relative to the total area of the biopolymer scaffold. For example, a 10×10 mm biopolymer scaffold patch may be provided having 20 through holes disposed therein and each of the through holes may be approximately 1 mm in diameter though other types of through holes with different sizes and distributions may also be used.

As noted above, in some embodiments, the through holes formed in a biopolymer scaffold material may be filled with a therapeutic composition (e.g., a growth factor) such that angiogenesis may direct the blood supply from neighboring non-ischemic regions of the heart to the through holes. In some embodiments, the through holes in the biopolymer scaffold material may vary in number, pattern, and/or size/diameter such that the therapeutic composition contained in the biopolymer scaffold material may vary between different portions of the biopolymer scaffold material. For example, areas of the biopolymer scaffold material with fewer through holes may hold a greater amount of a therapeutic composition within the biopolymer itself as compared to portions of the biopolymer scaffold material where larger numbers of through holes are formed. In such an embodiment, a therapeutic compound may be loaded in the perforated matrix first and then the cells may be added into the through holes of the biopolymer scaffold material. Without wishing to be bound by theory, the fewer the holes, the fewer the cells, but the more that other therapeutic compounds (e.g., drugs) may be loaded in the matrix.

In some embodiments, the through holes may be arranged to have a higher area density in select regions of the biopolymer scaffold material. For example, in some embodiments, the density of through holes may be greater in a portion of a biopolymer scaffold material intended to be positioned adjacent to an ischemic portion of a heart. Correspondingly, the area density of the through holes may decrease towards the edges of the material. In other embodiments, however, the highest density of through holes may be formed towards the edges of the material, and the area density of the through holes may decrease towards an interior of the material as the disclosure is not so limited.

The through holes may also be of any suitable transverse dimension (e.g., a width, diameter, or other appropriate type of dimension). In some embodiments, a suitable transverse dimension (e.g., width or diameter) of the through holes may be less than or equal to 1 mm in diameter, as through holes with this dimension have been observed by the inventors to stimulate and guide the growth of vasculature when a biopolymer scaffold material is applied to heart tissue. Without wishing to be bound by theory, the inventors have recognized that through holes with transverse dimensions of less than or equal to 1 mm may more effectively contain platelets, fibrin, and growth factors created by surgical bleeding in situ, thereby helping to promote vascularization in the ischemic region. In some embodiments, the through holes may also contain collagen borders that increase the surface area of the through holes such that platelets formed in the ischemic region may be activated due to greater contact area between the platelets and through holes, thereby leading to gel formation and greater blood retention. In view of the above, through holes formed in a biopolymer scaffold material of any of the embodiments disclosed herein may have an average maximum transverse dimension in a plane of a patch of the material (e.g. a width or diameter) that is less than or equal to 1.0 mm, 0.9 mm, 0.8 mm, 0.7 mm, 0.6 mm, 0.5 mm, 0.4 mm, 0.2 mm, 0.1 mm, and/or any other appropriate dimension. The through holes may also have an average maximum transverse dimension that is greater than or equal to 0.05 mm, 0.1 mm, 0.2 mm, 0.3 mm, 0.4 mm, 0.5 mm, and/or any other appropriate dimension. Combinations of the foregoing are contemplated including, for example, through holes with an average maximum transverse dimension that is between or equal to 0.05 mm and 1.0 mm. Other combinations of the foregoing dimensions as well as other appropriate dimensions are contemplated as the disclosure is not limited in this fashion.

As used herein, a through hole may refer to any passage that extends in an approximately linear fashion between two opposing surfaces of a biopolymer scaffold material (e.g., between two opposing surfaces of a patch formed from the biopolymer scaffold material). Depending on the embodiment, a through hole may include: slits formed in the biopolymer scaffold material where the material has been cut without removing material from the matrix such that the sides of the slit may still be proximate to one another when the material is in an unbiased state; cutouts where material has been removed from the biopolymer scaffold material to form a void extending between the opposing surfaces of the matrix; combinations of the forgoing; and/or any other appropriate type of through hole. It should be understood that the slits may have any appropriate shape within a plane parallel to a surface of the biopolymer scaffold material. For example, the slits may have a linear shape, a curved shape, other non-linear shapes, combinations of the forgoing, and/or any other appropriate type of shape. With regards to cutouts from the biopolymer scaffold material, the cutouts may have any appropriate type of cross-sectional shape in a plane parallel to a surface of the biopolymer scaffold material including, for example, squares, circles, rectangles, and/or any other appropriate type of cross-sectional shape. The orientations of the slits, cutouts, and/or other through holes may also be the same throughout an area of a biopolymer scaffold material such that the through holes are parallel to one another and/or the orientations may vary across the biopolymer scaffold material (e.g., switching between two or more orientations). Additionally, different types and/or shapes of through holes may be used in combination with one another. For instance, in some embodiments, a plurality of slits and a plurality of cutouts may be interspersed with one another. In yet another embodiment, a through hole may correspond to overlapping individually formed through holes. This may include overlapping slits, a slit overlapping with a cutout, overlapping cutouts, and/or any other appropriate configuration. Accordingly, it should be understood that the through holes disclosed herein should not be limited to any specific type of shape and/or arrangement.

In the various embodiments disclosed herein, a biopolymer scaffold material may be provided in the form of a patch, as discussed above. The patch may have a patch body with a planar shape that is substantially larger in the width and/or length dimensions as compared to a thickness of the patch body. The patch body may also be relatively flexible such that it may be draped over and/or otherwise conform to the shape of a structure it is attached to during a surgical procedure.

The various embodiments of biopolymer scaffold materials disclosed herein may take the form of a variety of suitable shapes, and the shapes may be varied depending upon the application. For example, the biopolymer scaffold material may be provided in the form of a patch which may have any suitable overall shape including, but not limited to a square, a rectangle, a circle, a non-regular closed geometric shape, or any other suitable shape. The biopolymer scaffold material may also have a shape that is symmetrical or asymmetrical depending on the application. In some embodiments, the shape of the biopolymer scaffold material may be customized such that the biopolymer scaffold material is substantially matched to the size and/or shape of an ischemic tissue region and a surrounding healthy tissue region of heart tissue of a patient that the biopolymer scaffold material may be implanted into. In some such embodiments, the biopolymer scaffold material size and/or shape may be determined from preoperative and intraoperative imaging of the patient. Thus, it should be understood that a biopolymer scaffold material may be provided in any appropriate size and/or shape for a desired application.

In some embodiments, the biopolymer scaffold materials may also be employed for use in locally delivering therapeutic compositions to heart tissue including, for example, myocardial tissue. This may include therapeutic compositions such as the growth factors noted above. A variety of other suitable types of therapeutic compositions may be used, as discussed in greater detail below. Regardless, in such an embodiment, the biopolymer scaffold materials may function as a reservoir for the therapeutic composition where the therapeutic composition is coated, bound to, absorbed, or otherwise loaded on or in the biopolymer scaffold material. The therapeutic compositions may be applied in the operating room during heart surgery and/or may be applied during manufacturing of the biopolymer scaffold material or any time prior to surgery as the disclosure is not so limited.

As used herein, the term “therapeutic composition” refers to a composition that is administered to a subject to treat a disease, disorder, or other clinically recognized condition, or for prophylactic purposes, and has a clinically significant effect on the body of the subject to treat, prevent, and/or diagnose the disease, disorder, or condition.

The variety of therapeutic compositions discussed herein may be delivered to a subject in a quantity greater than a trace amount to affect a therapeutic response in the subject. While the use of therapeutic compositions disclosed herein is predominantly discussed in reference to growth factors, a variety of therapeutic compositions may be used such as allogeneic cells, autologous cells, therapeutic agent-containing gels, allogeneic platelets, mRNA molecules, or any other suitable therapeutic compositions. In some embodiments, the autologous cells may be from native tissue, and may include stem cells (e.g., mesenchymal stem cells (MSCs)) or differentiated cells (e.g., cardiomyocytes). The aforementioned therapeutic compositions may be preloaded in or on the decellularized extracellular matrix prior to being implanted. In other embodiments, however, the therapeutic composition (e.g., cells) may be applied in or on the decellularized extracellular matrix of a biopolymer scaffold material once it is implanted in vivo. Of course, in some embodiments, therapeutic compositions can also include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. In another example, useful or potentially useful within the context of certain embodiments are compounds or chemicals traditionally regarded as drugs, vaccines, and biopharmaceuticals. Certain such therapeutic compositions may include molecules such as proteins, peptides, hormones, nucleic acids, gene constructs, cells (e.g., autologous cells, allogeneic cells, and/or any other appropriate type of cell), etc., for use in therapeutic, diagnostic, and/or enhancement areas. In certain embodiments, the therapeutic composition is a small molecule and/or a large molecule. Accordingly, it should be understood that the therapeutic compositions described herein are not limited to any particular type of therapeutic composition.

To facilitate absorption of a desired therapeutic composition into a matrix of a biopolymer scaffold material, in some embodiments, a therapeutic composition may be dissolved, dispersed, and/or otherwise mixed with a carrier liquid. Appropriate types of carrier liquids may include, but are not limited to water; saline; polysorbate; alcohol including benzyl alcohol, methanol, ethanol, or other appropriate type of alcohol; plasma; serum; other bodily fluids, and/or any other appropriate type of carrier. Of course, embodiments in which a carrier liquid is not used are also contemplated.

The characteristics of biopolymer scaffold materials described herein may make them especially suited for delivering growth factors and/or a variety of other therapeutic compositions to the heart when being used to aid in vascularization of an ischemic portion of a heart. However, it should be noted that the biopolymer scaffold material may be made of any material that provides the desired combination of properties described herein. Accordingly, the biopolymer scaffold materials described herein may have any suitable thickness, porosity, pore size, tortuosity, collagen fiber architecture, mechanical strength, biochemistry, surface properties, and/or other appropriate material parameters, as the disclosure is not so limited

While any appropriate type of biopolymer scaffold material may be used in the various embodiments disclosed herein, in some embodiments, the biopolymer scaffold may be a decellularized extracellular matrix. In some such embodiments, the decellularized extracellular matrix may comprise xenogeneic fetal or neonatal tissue. For example, in some embodiments, the biopolymer scaffold may be a bovine extracellular matrix (EBM) such as those under the tradename CardiaMend™. In other embodiments, the biopolymer scaffold may be a porcine extracellular matrix. Bovine extracellular matrix, porcine extracellular matrix, or other appropriate biopolymer scaffold from any other appropriate source may be decellularized in some embodiments to remove the allogenic and/or xenogeneic cellular antigens from the scaffold to reduce and/or prevent an immune response to the scaffold. In some embodiments, the biopolymer scaffold material may be made using fetal (i.e., prebirth) or neonatal bovine dermis that is less than 10 weeks of age post birth, 26 weeks of age post birth, and/or 52 weeks of age post birth. The use of bovine dermis may be especially advantageous in some embodiments due to the thickness, pore structure, and other parameters that aid in stimulating and guiding the growth of vasculature when applied to an ischemic portion of a heart. In some embodiments, the biopolymer scaffold material may be made using adult porcine dermis or neonatal porcine dermis that is less than 10 weeks of age post birth, 26 weeks of age post birth, and/or 52 weeks of age post birth to provide a desired material thickness. Without wishing to be bound by theory, selecting tissue from animals of different ages within the above ranges may permit the formation of different biopolymer scaffold materials with different thicknesses, porosity, mechanical strength, collagen fiber architecture, and/or other desirable parameters.

Generally, EBM is a biopolymer scaffold material derived from fetal, neonatal or post-natal animal tissue. EBM is processed in a way that preserves its tissue architecture and inherent strength characteristics without compromising the ability of cells to repopulate it and to remodel it. EBM may be used as a tissue-building component with or without cells for creating human body replacements.

According to some embodiments, EBM is produced from animal tissue by a method comprising the following steps: (1) removing the tissue from its source; (2) removing undesired cells, proteins, lipids, nucleic acids, and carbohydrates via chemical methods such as sodium chloride, hydrogen peroxide, sodium hydroxide, water and other optional solvents or chemicals; optionally extracting growth and differentiation factors from the tissue; (3) inactivating infective agents of the tissue; (4) mechanically expressing undesirable components from the tissue; (5) washing the tissue for removal of chemical residues; (6) optionally drying via lyophilization, supercritical CO2, air-drying, or other method; and (7) optionally cross-linking the tissue after chemical and mechanical treatment; and (8) optionally terminally sterilizing. As noted above, in some embodiments, EBM is made using fetal or neonatal bovine dermis that is any appropriate age that is less than 52 weeks of age, though embodiments in which tissue from an older animal is used is also contemplated. Again the tissue used to form the desired biopolymer scaffold material may be bovine dermis, porcine dermis, or any other appropriate tissue and/or source as the disclosure is not so limited. These manufacturing processes are further described in U.S. Pat. No. 9,011,895 which is incorporated herein by reference in its entirety for all purposes.

In some applications, it may be desirable to provide a particular collagen fiber architecture that is close to the collagen fiber architecture of the cardiac tissue that a biopolymer scaffold material is applied to. Accordingly, in some embodiments, the biopolymer scaffold material may include the following ranges and types of collagens. In some embodiments, the collagen contained in the matrix may be a native, intact, and/or non-denatured collagen from the original base matrix material. A biopolymer scaffold material, such as EBM, may include type I collagen in a dry weight percentage relative to the overall weight of the scaffold material that is greater than or equal to 60 wt %, 70 wt %, 75 wt %, 80 wt %, 90 wt % and/or any other appropriate range. The weight percentage of the type I collagen may also be less than or equal to 96%, 95%, 90 wt %, 85 wt %, 80 wt %, and/or any other appropriate range. The biopolymer scaffold material may also include a large quantity of type III collagen in a dry weight percentage relative to the overall weight of the scaffold material that is greater than or equal to 4 wt %, 5 wt %, 10 wt %, 15 wt %, 20 wt %, and/or any other appropriate range. Correspondingly, the weight percentage of type III collagen may be less than or equal to 40 wt %, 30 wt %, 25 wt %, 20 wt %, 10 wt %, and/or another appropriate range. Combinations of the above are contemplated including, for example, type I collagen in a range between or equal to 60 wt % and 95 wt % and type III collagen in a range between or equal to 5 wt % and 40 wt %, or more preferably 60 wt % to 80 wt % type I collagen as well as 20 wt % to 40 wt % type III collagen, may correspond to material formed from fetal and neonatal dermis. Correspondingly, type I collagen in a range around about 95 wt % and type III collagen around about 5 wt % may correspond to adult dermis. This difference in collagen type content may lead to different material properties. Of course, the inclusion of other types of collagens as well as weight percentages different than those noted above are also contemplated as the disclosure is not limited in this fashion.

The biochemistry of EBM makes it an ideal candidate as a biopolymer scaffold material for use in tissue repair. EBM does not incite significant inflammation because the manufacturing process does not significantly damage the native collagen fibers physically or biochemically. EBM may also be substantially free of xenogeneic growth factors that incite inflammation. In applications where EBM is used to provide a growth factor or a variety of therapeutic compositions to the myocardial tissue, EBM may not damage, injure, or otherwise further exacerbate any trauma to the myocardial tissues because it may be placed on the epicardium and/or pericardium in some embodiments rather than placed directly on the heart. EBM may also be devoid of measurable quantities of xenogeneic growth factors or extracellular matrix proteins that may cause inflammation which other scaffold materials may retain.

Another benefit of EBM is that it can be made in various thickness configurations that may be difficult, or impossible to practically obtain using other scaffold materials. In some embodiments, the unique fetal and neonatal bovine source allows EBM to be provided in a wide range of thicknesses and area ranges. In contrast, typical porcine dermis (another acellular dermal matrix not processed with the above noted processing techniques) uses horizontal splitting (cutting) to get uniformly thick materials which limits the thickness of this matrix which also includes different pore, mechanical, and biochemical properties. In contrast, small intestinal submucosa (SIS) and urinary bladder matrix materials inherently exhibit low-porosity, low absorption capacity, and low absorption rate in addition to being thinner materials that need several layers to be laminated together to provide increased thicknesses which impacts the internal properties of the matrix, and are typically provided with thicknesses less than 1 mm.

Benefits associated with increased thicknesses of the scaffold materials disclosed herein may include, for example, increased reservoir volumes for containing therapeutic compositions for long-duration release. Thus, in some embodiments, EBM, or other appropriate scaffold material, may have a thickness that is greater than or equal to about 0.2 mm, 0.5 mm, 1 mm, 2 mm, 3 mm, 4 mm, 5 mm, and/or any other appropriate thickness. Additionally, the thickness of EBM or other biopolymer scaffold materials may be less than or equal to 6 mm, 5 mm, 4 mm, 3 mm, 2 mm, 1 mm, and/or any other appropriate thickness. Combinations of these thicknesses are contemplated including, for example, a scaffold thickness that is between or equal to 0.2 mm and 6 mm. Of course, thicknesses both greater than and less than those noted above are also possible.

Other benefits of EBM is its absorption rate and absorption capacity. EBM may be lyophilized, resulting in a highly porous membrane that can rapidly absorb large quantities of therapeutic compositions, which in some embodiments may be suspended in a carrier, even when provided in a thickness greater than a 1 mm thick configuration. Furthermore, EBM may be lyophilized at the end of manufacture, which, in combination with the thickness range of the device, leads to a device with extremely high liquid absorption characteristics. Thus, in some embodiments, a biopolymer scaffold matrix, such as EBM, may have an areal absorption capacity that is greater than or equal to 0.1 ml/cm², 0.2 ml/cm², 0.3 ml/cm², 0.4 ml/cm², 0.5 ml/cm², and/or any other appropriate absorption capacity (herein areal absorption capacity means volume of fluid per square area of EBM, however effective absorption capacity means volume of fluid per volume of EBM) and, depending on the growth factor and/or therapeutic composition, may be saturated in less than 10 minutes. The areal absorption capacity may also be less than or equal to 1.0 ml/cm², 0.9 ml/cm², 0.8 ml/cm², 0.7 ml/cm², 0.6 ml/cm², and/or any other appropriate absorption capacity. Combinations of these ranges are contemplated including, for example, an areal absorption capacity that is between or equal to 0.1 ml/cm² and 1.0 ml/cm².

In contrast to the above, other scaffold materials may be thin compared to EBM and therefore incapable of absorbing these relatively large amounts of a therapeutic composition. For example, SIS is approximately 0.2 mm to 0.4 mm in thickness such that SIS would need multiple bonded layers to reach similar thicknesses, and even then, such a construction is unlikely to provide similar benefits due to the bonding process and inherent properties of the SIS itself. For instance, other materials, such as non-dehydrated materials like porcine acellular dermal matrix (e.g., Strattice) and SIS are either provided wet or hydrated, are dense with relatively little porosity, and/or have other limitations such that they do not allow for the device to hold such high loadings of a therapeutic composition and/or to be quickly soaked like a sponge in the operating room. That said, any appropriate biopolymer scaffold material exhibiting a desired combination of properties may be used as the disclosure is not limited in this fashion.

Due to the large source material, EBM, or other biopolymer scaffold materials as described herein, may be provided with a range of areas and/or shapes for various applications including any of the applications described previously above. Depending on the specific application, appropriate sizes for the scaffold material may include areas that are greater than or equal to 1 cm², 20 cm², 50 cm², and 100 cm², 150 cm², 200 cm², and/or any other appropriate size. The size of the scaffold material may also include areas that are less than or equal to 200 cm², 150 cm², 100 cm², 50 cm², 20 cm², and/or any other appropriate size. Combinations of the foregoing are contemplated including, for example, scaffold materials with areas that are between or equal to 1 cm² and 200 cm². Specific exemplary patch sizes that may be used in some applications may include, but are not limited to, 2×2 cm, 3×3 cm, 4×4 cm, 5×6 cm, 6×12 cm, 7×15 cm, 8×16 cm, 10×15 cm, or 10×20 cm. Of course, while specific size ranges are provided above, it should be understood that any appropriate size biopolymer scaffold material may be used including areas both greater than and less than those noted above as the disclosure is not limited in this fashion.

In some embodiments, EBM using bovine sources may also have superior mechanical properties as compared to other typical biopolymer scaffold materials such as porcine dermis and SIS. For example, it is a strong, yet elastic material. By selecting for age, without splitting, and by nature of the collagen fiber architecture unique to the bovine source, EBM is extremely strong yet remains soft and pliable with stiffness similar to other human soft tissues (unlike most synthetic polymers, metals, or ceramics) including the pericardium and myocardium. Thus, EBM based devices are mechanically strong (stronger than other scaffold materials such as SIS) such that they provide stable pericardial reconstruction and support. Additionally, EBM based devices may be compliant and soft such that they do not significantly affect heart function or result in mechanical rubbing and/or abrading of the myocardium when contacting the heart during normal sinus rhythm. Of course, while EBM may be preferable in some applications, it is expected that a biopolymer scaffold material made using porcine dermis and the above noted processing techniques will also exhibit superior absorption, release, thickness, and strength as compared to typical SIS materials.

In view of the above, a biopolymer scaffold material as disclosed herein, including an EBM, may have an ultimate tensile strength that is greater than or equal to 1 MPa, 3 MPa, 5 MPa, 10 MPa, 20 MPa, 30 MPa, 40 MPa, and/or any other appropriate tensile strength. The ultimate tensile strength may also be less than or equal to 60 MPa, 50 MPa, 40 MPa, 30 MPa, and/or any other appropriate tensile strength. Combinations of foregoing are contemplated including, for example, an ultimate tensile strength of a biopolymer scaffold material may be between or equal to 5 MPa and 60 MPa. Of course, tensile strengths both greater than and less than those noted above are also contemplated as the disclosure is not so limited.

A biopolymer scaffold material, such as EBM, may also have an improved suture pullout as compared to other materials. For example, a suture retention strength of the biopolymer scaffold material may be greater than or equal to 5 N, 10 N, 20 N, 50 N, 100 N, 200 N, 300 N, and/or any other appropriate suture retention strength. The suture retention strength of the biopolymer scaffold material may also be less than or equal to 500 N, 400 N, 300 N, 200 N, 100 N, and/or any other appropriate suture retention strength. Combinations of the foregoing are contemplated including, for example, a suture retention strength that is between or equal to 10 N and 500 N. Of course, suture retention strengths both greater than and less than those noted above are also contemplated as the disclosure is not limited in this fashion. The above-noted suture retention strengths may be measured using a suture having a thickness equivalent to USP suture size/diameter for the intended procedure (4-0-2) during a standard suture pull out test.

In some instances, and as noted above, it may be desirable for a biopolymer scaffold material, such as EBM, to exhibit a desired amount of elasticity for cardiac applications. Accordingly, in some embodiments, a Young's modulus, sometimes referred to as an elastic modulus, of the biopolymer scaffold material may be greater than or equal to 1 MPa, 3 MPa, 10 MPa, 20 MPa, 30 MPa, 40 MPa, 50 MPa, 100 MPa, 200 MPa, and/or any other appropriate range. The Young's modulus may also be less than or equal to 400 MPa, 300 MPa, 200 MPa, 100 MPa, 50 MPa, 40 MPa, 30 MPa, and/or any other appropriate range. Combinations of the foregoing are contemplated including, for example, between or equal to 1 MPa and 400 MPa as well as between or equal to 20 MPa and 200 MPa. Of course, while specific ranges are provided, ranges both greater than and less than those noted above are also contemplated as the disclosure is not limited in this fashion.

Another benefit of EBM is the tortuous interconnected open pore structure of the material. Thus, a biopolymer scaffold material may include a plurality of interconnected open pores that connect a first surface of the biopolymer scaffold material to an interior portion of the biopolymer scaffold material, and in some embodiments, a second opposing surface of the biopolymer scaffold material. A flow path extending through the plurality of interconnected pores may follow a tortuous, i.e., non-linear, path such that a liquid may flow into and subsequently out of the scaffold material during loading and eluting phases of the material. In some examples, the interconnected tortuous porosity of EBM is provided by the crisscrossing collagen fiber architecture of EBM. This porosity and architecture allow fluid to be absorbed quickly in a surgical setting (e.g., minutes) while still acting as a barrier against inflammation inducing molecules and/or providing the desired therapeutic composition elution properties.

A biopolymer scaffold material, such as EBM, used in the embodiments disclosed herein may have a porosity that is greater than or equal to 30%, 40%, 50%, and/or other appropriate porosity. The porosity may also be less than or equal to 80%, 70%, 60%, 50%, and/or any other appropriate porosity. Combinations of foregoing are contemplated including, porosities that are between or equal to 30% and 80%. However, porosities both greater than and less than those noted above are also contemplated as the disclosure is not so limited. Additionally, without wishing to be bound by theory, the high porosity nature of the materials disclosed herein may affect the overall volume and areal capacities of the materials.

A biopolymer scaffold material, such as EBM, used in the embodiments disclosed herein may have an average pore size that is greater than or equal to 1 μm, 2 μm, 5 μm, 10 μm, 50 μm, 100 μm, 250 μm, and/or any other appropriate size. The average pore size may also be less than or equal to 500 μm, 250 μm, 100 μm, 50 μm, and/or any other appropriate size. Combinations of foregoing are contemplated including, for example, an average pore size of a biopolymer scaffold material that is between or equal to 1 μm and 500 μm. Of course, average pore sizes both greater than and less than those noted above are also contemplated.

Due to the collagen fibers swelling and shrinking depending on the exposure of the biopolymer scaffold materials to a given liquid, the above porosities and average pore sizes may be measured in the dry state prior to implantation and prior to introduction of a carrier liquid and/or therapeutic composition to the biopolymer scaffold material. Additionally, the pore sizes and porosity may be measured using microscopic optical image analysis.

In some embodiments, it may be desirable to improve the wicking capabilities of a biopolymer scaffold material. Such a modification may improve the ability of the biopolymer scaffold material to absorb liquids, may alter the elution kinetics of a therapeutic compound from the scaffold material, and/or may help to reduce the creation of unfilled occluded portions of the material due to the inclusion of air pockets in the matrix. Accordingly, in some embodiments, it may be desirable to include hydrophilic modifications and/or surface coatings within the pores of the biopolymer scaffold material. In such an embodiment, the surface modification and/or coating on the surface of the pores of the biopolymer scaffold material may be more hydrophilic, i.e., exhibit a lower water contact angle, than the underlying biopolymer scaffold material itself. Appropriate types of hydrophilic modifications and/or coatings may include, but are not limited to polyethylene glycol (PEG), crosslinked collagen; degradable polymers such as poly(lactic acid) (PLA), poly(glycolic acid) (PGA), and P4HB; and/or permanent polymers. Of course, embodiments in which a hydrophilic modification and/or coating are not used are also contemplated.

As described above, a biopolymer scaffold material, such as EBM, acts as a reservoir for a therapeutic composition such as a growth factor or other desired therapeutic compositions. When the scaffold material containing a therapeutic composition is attached to heart tissue in a subject, the device may locally deliver the therapeutic composition from the scaffold material to adjacent tissues and/or other tissues located adjacent to and/or downstream from the scaffold material. Another benefit of the disclosed biopolymer scaffold materials, including EBM, are the therapeutic composition release characteristics that allow for therapeutic composition delivery in a specified therapeutic window. In some embodiments, the therapeutic compositions may not bind strongly to the collagen of the device. Also, the porosity and fluid flow characteristics of a biopolymer scaffold material may slow down the release of the therapeutic composition as compared to a bolus or simple injection delivery to the pericardial fluid. Since this release of the therapeutic composition is local, it may reduce the systemic load of a growth factor, or other therapeutic composition, to other organs and tissues.

It should be understood that while the properties and characteristics for a biopolymer scaffold material provided above and elsewhere in the current disclosure are primarily described relative to EBM, these properties and characteristics may be found in other biopolymer scaffold materials as well. For example, decellularized porcine extracellular matrix materials and/or other appropriate materials prepared using the methods described herein may exhibit the properties and characteristics described herein.

According to other embodiments, the inventors have also recognized the benefits associated with forming microchannels that extend at least partially through the heart tissue of ischemic portions of a heart, and in some instances, healthy portions of the heart surrounding the ischemic portions of the heart. In such an embodiment, the microchannels may extend through an epicardium of the heart and partially through the myocardium of the heart by a controlled depth. In other embodiments, however, the microchannels may extend through the entire heart muscle as will be discussed in greater detail below. Thus, the microchannels formed in the heart tissue may or may not extend through the endocardium of a subject's heart. This may serve to limit the damage to the heart tissue while still improving vascularization in the ischemic region of the heart by promoting angiogenesis from neighboring regions of healthy tissue. Additionally, in some embodiments, at least a portion of the microchannels formed in the heart tissue of a subject may be interconnected which may help to further improve blood flow and vascularization in ischemic heart tissue via a microchannel network that extends at least partially in a direction that is effectively parallel to the surface of the epicardium. In some such embodiments, the interconnected microchannel network may be formed such that the microchannels extend from a non-ischemic region of the heart tissue to an ischemic region of heart tissue to promote vascularization in the ischemic region. In contrast, TMR, as traditionally employed and described above, only fully penetrates through the endocardium of a subject's heart and into the ventricle, which may lead to excessive bleeding and can damage certain regions of tissue while not exhibiting demonstrable benefits with regards to vascularization of the ischemic heart tissue. In addition to promoting vascularization, the use of microneedles to form microchannels may result in additional benefits such as denervation of the myocardium, thereby serving to reduce or prevent the adverse health effects of heart conditions such as angina.

In some embodiments, the inventors have found benefit in forming microchannels that extend through the entire heart muscle (i.e., past the endocardium and into the ventricle of the heart). In addition, as noted above, at least a portion of the microchannels formed in heart tissue may be interconnected which may further help to improve blood flow and promote vascularization. In some such embodiments, the inventors have recognized that by extending microchannels formed in the heart into the ventricle, high pressure ventricular blood may circulate through the interconnected network of microchannels, thereby increasing blood supply to ischemic regions of heart tissue. While the microchannels may extend into the ventricle in embodiments where an interconnected network of microchannels is formed, the microchannels may also extend into the ventricle in embodiments where an interconnected network of microchannels is not present, as the disclosure is not so limited.

The above noted microchannels may be formed in heart tissue using any suitable method. For example, in some embodiments, the microchannels may be formed individually and/or in a sequential pattern. In other embodiments, however, the microchannels may be formed in a single stroke such that each of a plurality of microneedles penetrate the heart tissue in a simultaneous manner. In some embodiments, the microchannels may be formed such that they are substantially aligned or non-aligned to corresponding through holes on a biopolymer scaffold material, as will be discussed in greater detail below. Similarly, in some embodiments, the plurality of microneedles may be arranged such that the microneedles are substantially aligned or non-aligned with the through holes on the biopolymer scaffold material, as discussed further below. The microchannels may also be formed using static microneedles. Alternatively, the microchannels may be formed using a device with one or more microneedles that may be extended between an extended configuration and a retracted configuration to penetrate partially through the myocardium of a subject's heart. For example, in some embodiments, a plurality of microneedles of a device may be movable between a retracted configuration and an extended configuration to penetrate either ischemic and/or non-ischemic portions of the heart tissue when transitioned into the extended configuration to form microchannels in these regions. Of course, any appropriate method for forming microchannels that extend through the epicardium and partially through the myocardium of a subject's heart without puncturing the endocardium may be used as the disclosure is not so limited. This may include for example, controlled laser ablation, radiofrequency (RF) ablation, Ultrasound (US) ablation, and/or other appropriate methods.

The microchannels formed in the heart tissue of a subject may be formed in any appropriate way and may have any appropriate shape. For example, in some embodiments, the microneedles may be configured as pointed needles that penetrate the epicardium and extend into the myocardium, but do not remove any heart tissue. In other embodiments, the microneedles may be configured as coring needles that penetrate the epicardium and extend into the myocardium, where the coring needles remove a portion of heart tissue approximately equal to an inner diameter and shape of an interior cannula of the coring needles.

The inventors have recognized that, in some embodiments, benefits may be realized by forming microchannels that are selectively patterned to increase heart perfusion through promoting directed vascularization from non-ischemic regions of heart tissue to ischemic regions of heart tissue. This may be achieved by selectively choosing characteristics of the microchannels such as the depth, direction, area density, size, shape, and/or degree of interconnectivity between adjacent microchannels. The formation of these patterns of microchannels may be guided by an end user (e.g., a surgeon), a surgical robot, a microchannel patterning device, and/or any other appropriate method. In some embodiments, the patterns may be directly visualized by the end user, computer guided, or pre-planned based on preoperative and/or intraoperative imaging of the heart perfusion. Such imaging may include, but is not limited to magnetic resonance imaging (MRI).

The inventors have also recognized that the appropriate patterns and microchannel characteristics may also vary from patient to patient. For example, each of pediatric, adult, and geriatric patient groups may have different heart tissue thicknesses that influence the depth of the formed microchannels to penetrate into the myocardium without penetrating the endocardium of the heart. Further, the heart tissue may also have different thicknesses depending on the location of the heart tissue and ischemic portions of a heart may vary from subject to subject. Thus, the ischemic portions of a heart that are to be treated as well as the heart tissue thickness that is to be partially penetrated may be determined for a given patient through use of preoperative and intraoperative imaging methods as disclosed above, such as magnetic resonance imaging (MRI).

In view of the above, appropriate length microneedles and/or microchannels may be selected to penetrate through the epicardium and partially through a myocardium of a subject without penetrating through the endocardium of the subject based on a determined thickness of a subject's heart tissues in a region in which microchannels are to be formed. In some embodiments, an appropriate length of a microchannel and/or a corresponding length that a microneedle may extend into tissue in an extended, or other appropriate configuration, may be greater than or equal to 1 mm, greater than or equal to 5 mm, greater than or equal to 6 mm, greater than or equal to 7 mm, greater than or equal to 8 mm, greater than or equal to 9 mm, greater than or equal to 10 mm, or any other appropriate length. Accordingly, the length of the microneedles and/or microchannels may be less than or equal to 15 mm, less than or equal to 14 mm, less than or equal to 12 mm, less than or equal to 10 mm, less than or equal to 5 mm, and/or any other appropriate distance. Combinations of the above ranges appropriate to provide microneedles and/or microchannels that extend through the epicardium and into the myocardium, but not through the endocardium of the patient's heart are contemplated including lengths that are between or equal to 5 mm and 15 mm, 8 mm and 12 mm, and/or any other appropriate length.

While a variety of types of microneedles have been disclosed above, any suitable type of microneedle may be used including, but not limited to pointed needles, angled needles, coring needles, hooked needles, or any other suitable needle type. The microneedles may also be of any suitable size to provide the desired benefits associated with reperfusion and vascularization of ischemic heart tissue. For example, the microneedles and resulting microchannels may have average maximum transverse dimensions (e.g., outer diameter) that is perpendicular to a longitudinal axis of the microneedles and/or microchannels that is greater than or equal to 0.1 mm, greater than or equal to 0.2 mm, greater than or equal to 0.3 mm, greater than or equal to 0.4 mm, greater than or equal to 0.5 mm, greater than or equal to 0.6 mm, greater than or equal to 0.7 mm, greater than or equal to 0.8 mm, greater than or equal to 0.9 mm, greater than or equal to 1.0 mm, or any other appropriate transverse dimension. Correspondingly, the average maximum transverse dimension of the microneedles and/or microchannels may be less than or equal to 1.0 mm, 0.9 mm, 0.8 mm, 0.7 mm, 0.6 mm, 0.5 mm, and/or any other appropriate dimension. Combinations of the forgoing are contemplated including, for example, an average maximum transverse dimension of the microneedles and/or microchannels that is between or equal to 0.1 mm and 1.0 mm. Without wishing to be bound by theory, this range of microchannel sizes have been observed to be associated with improved perfusion and revascularization of heart tissue treated in this manner.

While any of the above noted methods and devices may be used to improve perfusion and vascularization of ischemic heart tissue, these separate therapeutic concepts may interact with one another to provide synergistic benefits that provide improved perfusion and vascularization of heart tissue beyond a simple addition of their separate performances. For example, the inventors have recognized that benefits may be realized by providing both a biopolymer scaffold material (e.g., an EBM patch) having a plurality of through holes formed therein and a plurality of microchannels formed in the ischemic heart tissue that the biopolymer scaffold material is applied to. Such a combination may specifically help in directing angiogenesis and enhancing heart vascularization in an ischemic region of heart tissue. Further, in some embodiments, the combination of a biopolymer scaffold material with a plurality of microchannels formed in the underlying heart tissue may serve to provide a vascular bed for delivering and/or supporting delivered cells to an ischemic region of heart tissue. Specifically, the inventors have recognized that such a configuration may promote survival of cells that may be supported on or loaded in the pores and/or through holes of the biopolymer scaffold material as previously described above. This may correspondingly reduce the number of cells needed to promote revascularization. This combination of devices and therapies using any of the disclosed methods for forming microchannels in heart tissue and any of the disclosed biopolymer scaffold materials may, thus, offer a unique therapy with unique benefits as compared to using either of these therapies individually.

In some embodiments, microchannels may be formed in the heart tissue prior to the application of a biopolymer scaffold material. In some such embodiments, through holes of the biopolymer scaffold material patch may be aligned with the pre-formed microchannels upon application of the EBM patch to the heart tissue surface. The inventors have recognized that benefits may be realized by aligning the through holes of the biopolymer scaffold material patch with the microchannels as this may promote vascularization originating from a non-ischemic portion of heart tissue to be directed into the through holes of the biopolymer scaffold material patch and in turn into the microchannels formed in the ischemic region of the heart tissue. In other embodiments however, the through holes of the biopolymer scaffold material patch may not be aligned with the existing microchannels formed in the heart tissue.

It should be understood that the various embodiments disclosed herein pertaining to biopolymer scaffold materials, microneedling devices, therapeutic compositions, and/or combinations thereof may be used for any suitable applications including to treat ischemic regions of heart tissue. The embodiments disclosed herein may also be embodied as various devices and/or methods and may be arranged in any suitable combination as the disclosure is not so limited. Further, the embodiments of biopolymer scaffold materials and microneedling devices disclosed herein may be combined in any suitable arrangement as the disclosure is not so limited. In some embodiments, the biopolymer scaffold materials and microneedling devices may be provided in the form of a kit prepared for the end user (e.g., a surgeon). The kit may comprise a biopolymer scaffold material (e.g., in the form of a patch), a microneedling device, and/or one or more therapeutic compositions (e.g., a growth factor), among other components as the disclosure is not so limited.

As disclosed herein, the inventors have recognized that a variety of benefits may be realized by employing perforated porous biopolymer scaffold materials, by forming microchannels in heart tissue, and/or by employing the combination thereof to promote and guide angiogenesis, and enhance heart vascularization in ischemic regions of heart tissue. Such benefits may include, but are not limited to the formation of smaller microchannels relative to existing technologies which may serve to limit damage to the heart tissue, and improving reperfusion of the myocardium by directing angiogenesis from non-ischemic heart tissue to ischemic heart tissue to promote revascularization. Additional benefits may also include providing a device with both a biopolymer scaffold material and microchannel forming device (e.g., microneedling devices) in combination to maximize effective revascularization in a region of ischemic heart tissue, and/or providing possible regeneration of the heart tissue. However, benefits different from those noted above are also possible.

For the sake of clarity, the majority of embodiments referenced and discussed herein refer to the use and properties of decellularized bovine extracellular matrix (EBM) which may offer certain benefits when used to treat ischemic heart tissue. However, the noted properties and results should be understood as also being applicable to decellularized porcine extracellular matrix materials and/or other appropriate materials as the disclosure is not so limited. Additionally, while the majority of embodiments discussed herein refer to the use of growth factors to facilitate revascularization and/or regeneration of ischemic heart tissue, any suitable therapeutic composition may be employed as the disclosure is not limited in this regard.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

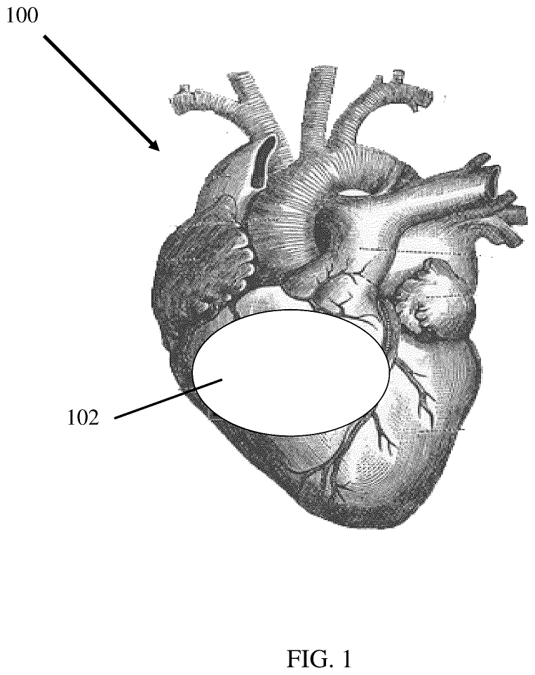

FIG. 1 shows an embodiment of a heart 100 of a subject having an ischemic portion 102 of the heart that includes ischemic heart tissue. The ischemic portion 102 may be of any size and/or shape, and may vary from patient to patient. As disclosed herein, the ischemic portion 102 may be present in the heart 100 of the subject due to limited perfusion of the myocardium, e.g., due to a myocardial infarction (MI) or progressive heart failure (HF).

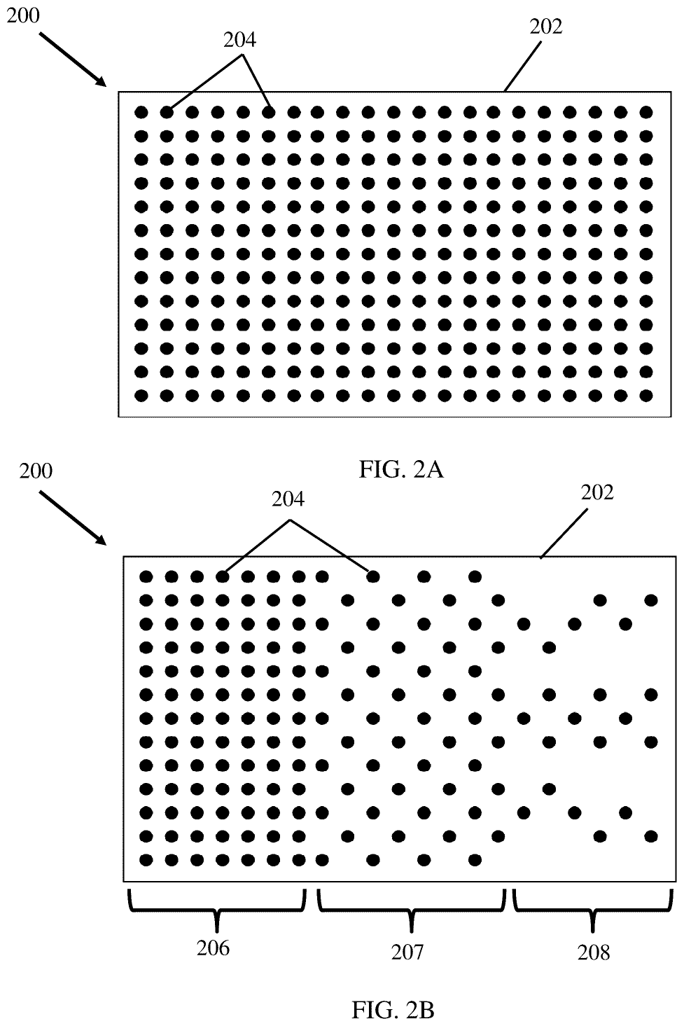

FIG. 2A-B show embodiments of a biopolymer scaffold material 200 in the form of an EBM patch 202 comprising a plurality of through holes 204 that pass from a first planar surface of the patch to a second opposing planar surface of the patch. In FIG. 2A, the EBM patch 202 has a uniform distribution of through holes 204 throughout the area of the patch. By contrast, in FIG. 2B, the EBM patch 202 has a non-uniform distribution of through holes 204 across the area of the patch such that a density and/or layout of the through holes may vary between two or more portions of the EBM patch, or other biopolymer scaffold material. Specifically, the EBM patch 202 embodied in FIG. 2B has a first section 206, a second section 207, and a third section 208 each having a different distribution of the plurality of through holes 204. As shown in FIG. 2B, the first section 206 has the greatest area density of through holes 204 arranged in a square grid layout, followed by the second section 207 having a lesser area density of through holes 204 in a diamond grid layout, and lastly the third section 208 has the lowest area density of through holes 204 and may not have a uniform distribution of the through holes in this section. Thus, it should be understood that any appropriate distribution, number, and/or layout of the through holes may be used as the disclosure is not so limited. As disclosed herein, the through holes 204 may serve to promote angiogenesis and guided vascularization to ischemic portions of the heart tissue when applied to the heart of a patient. In some embodiments, the through holes 204 may be actively filled with platelets, platelet-rich plasma (PRP), or platelet gel, one or more cell populations, or inactively filled via surgical bleeding in situ to promote vascularization.

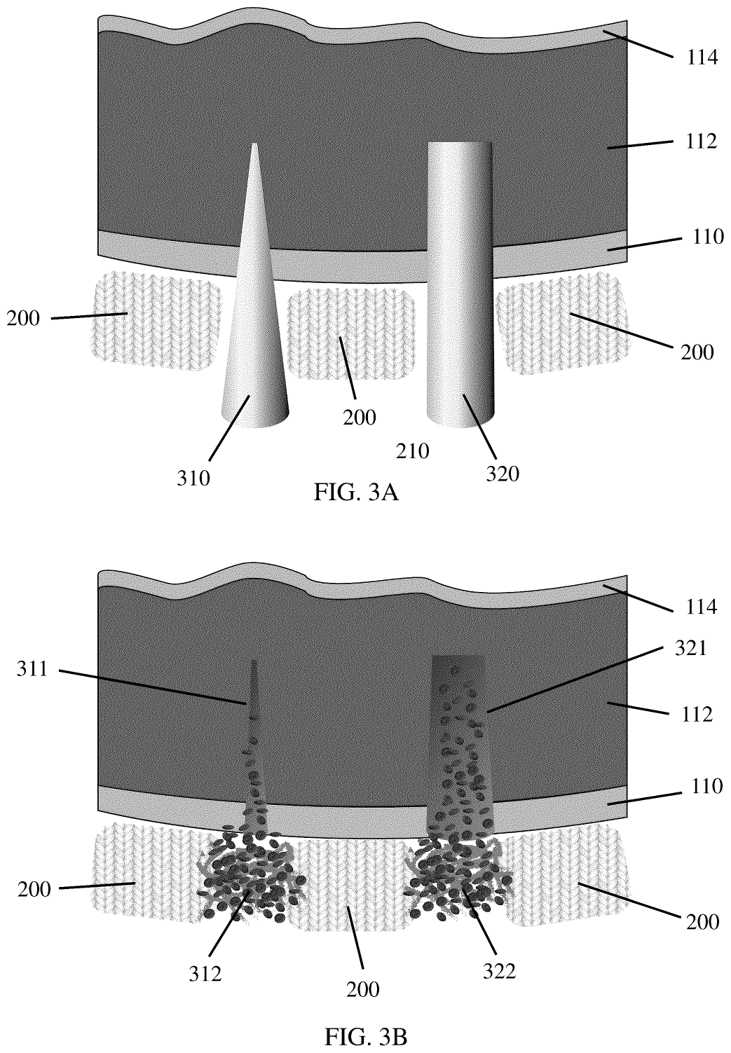

FIG. 3A shows an embodiment of a region of heart tissue where an EBM patch 200 has been applied to the epicardium 110 of an ischemic portion of a heart. Separately, one or more of a variety of microneedles 310 and 320 may be penetrated through the epicardium 110 and partially into the myocardium 112 of the heart of a patient. Specifically, FIG. 3A shows the microneedles 310 and 320 being extended through the through holes formed in the EBM patch 200 to extend into the patient's heart tissue. As shown, the microneedles 310 and 320 extend through the epicardium 110 and into the myocardium 112 of the patient, but not through the endocardium 114 and into the ventricle of the heart. In FIG. 3A, the microneedle 310 is shown as a pointed needle whereas the microneedle 320 is shown as a coring needle. The microneedles 310 and 320 may be extended any suitable distance into the myocardium 112 according to embodiments disclosed herein. Additionally, in this embodiment, the microneedles are shown as being aligned with the through holes of the EBM patch. However, as noted previously, microneedling may alternatively be done prior to application of the EBM patch such that the resulting microchannels may not be aligned with the through holes as the disclosure is not so limited. Additionally, different types of microneedles have been shown for illustrative purposes, though, either a single type or multiple types of microneedles may be used for therapeutic purposes as the disclosure is not so limited.

FIG. 3B shows an embodiment of FIG. 3A where the microneedles 310 and 320 have been removed from the heart tissue of the patient, thereby forming microchannels 311 and 321 with blood flows 312 and 322 flowing out from the myocardium towards the EBM patch 200, respectively. As shown, the pointed microneedle 310 may result in a microchannel 311 that has minimal or no tissue removed whereas the coring microneedle 320 may result in a microchannel 321 that removes a portion of the tissue approximately equal to the internal diameter of the coring needle. As disclosed herein, the formation of microchannels 311 and 321 may serve to connect the myocardium 112 to the corresponding through holes formed in the EBM patch 200 which may in turn promote vascularization of the heart tissue.

As noted above, it may be desirable to form interconnected guided vasculature in the heart tissue of a subject which may facilitate reperfusion of ischemic tissue. Accordingly, in some embodiments, the microneedles used to form microchannels in tissue may be configured to form overlapping guided vasculature, also referred to as interconnected guided vasculature herein, in the heart. Such overlapping vasculature may occur with any type of microneedle as disclosed herein. The inventors have found that such interconnected guided vasculature where a flow path may extend between multiple separately formed microchannels may further promote revascularization of the ischemic heart tissue by promoting blood flow.

In some embodiments, the microneedles may have a straight profile. Such straight microneedles may be used to create substantially parallel microchannels in the patient's heart tissue. In other embodiments, however, the microneedles may have a curved profile. In some such embodiments, the inventors have recognized that the curved microneedles may be used to connect areas of good perfusion (e.g., a non-ischemic area of the heart) to ischemic areas of the heart to promote vascularization. The use of a curved needle profile may also ensure that the microneedles do not extend past the endocardium and into the ventricle of a patient's heart. The curved microneedles may also be curved to any degree. For example, the curved microneedles may have a “U-shape”, or other appropriate curved shape, such that the microneedles may enter the heart tissue in a first direction and exit an adjacent portion of the heart tissue in a second at least partially opposing second direction. Any of the needle types described herein may exhibit any needle profile in any suitable combination as the disclosure is not so limited. For example, a pointed microneedle may have a straight or curved profile. In another example, a coring microneedle may have a straight or curved profile.

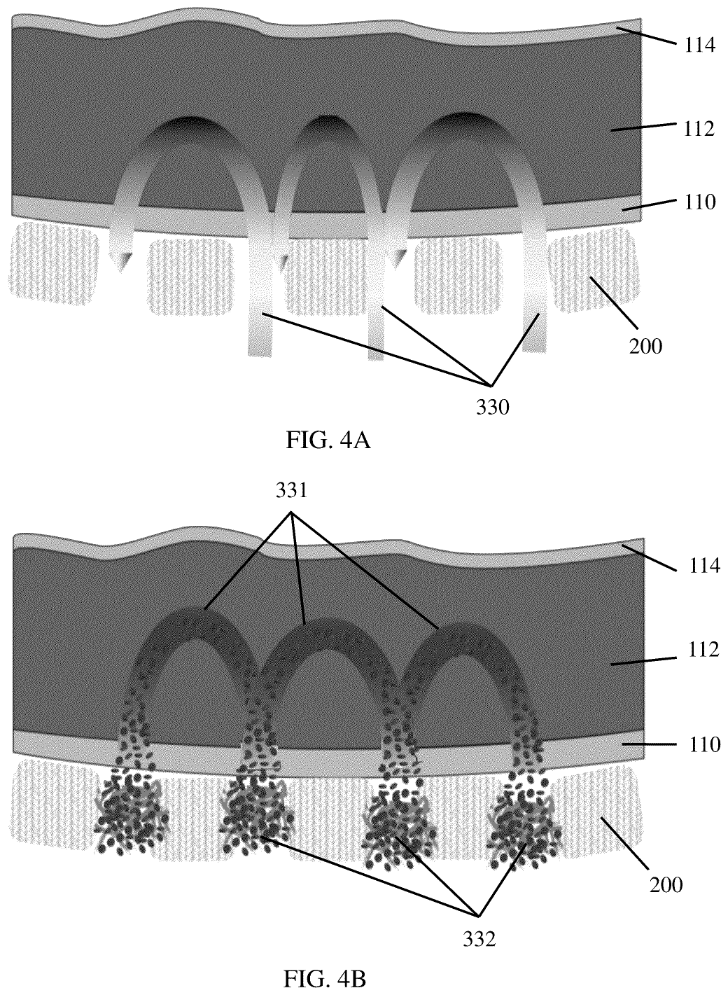

In view of the above, a plurality of microneedles having a curved profile may be inserted into a region of heart tissue such that the microneedles may enter the heart tissue in a first direction and exit an adjacent portion of the heart tissue in a second direction that may be at least partially opposite from the first. In such a configuration, the penetration of the microneedles having a curved profile may be repeated along the heart tissue, such that each substantially curved microchannel formed by the curved microneedles may be connected to one another to create interconnected curved guided vasculature to promote revascularization. Such an embodiment is illustrated in FIG. 4A which shows an embodiment of a region of heart tissue with an EBM patch 200 applied thereon and a plurality of curved microneedles 330 that have been penetrated into the myocardium 112 of the heart of a patient. Specifically, FIG. 4A shows the curved microneedles 330 being extended through the through holes formed in the EBM patch 200 to extend into the patient's heart tissue. As shown, the curved microneedles 330 extend through the epicardium 110 and into the myocardium 112 of the patient, but not through the endocardium 114 and into the ventricle. Rather, the curved microneedles 330 exit the epicardium 110 of the patient in an adjacent region of the heart tissue located to a side of the entry point or base of the microneedle and in a direction that is at least partially opposite from the initial direction of microneedle penetration. The curved microneedles 330 may be configured such that penetration of each curved microneedle 330 exits the epicardium 110 nearby to where an adjacent curved microneedle 330 initially penetrates the epicardium 110 and/or at a location where a microchannel is formed within the myocardium of the heart. Thus, the curved microneedles with overlapping deployment paths may be used to form interconnected guided vasculature in the myocardium 112. In FIG. 4A, the curved microneedles 330 are shown to be configured as coring needles, but any suitable needle type may be used such as pointed needles as the disclosure is not so limited.

FIG. 4B shows the embodiment of FIG. 4A after the curved microneedles 330 have been removed from the heart tissue of the patient, thereby forming microchannels 331 with blood flow 332. The resulting microchannels 331 may be at least partially interconnected with one another in the heart tissue as shown, thereby forming overlapping guided vasculature. As disclosed herein, the formation of interconnected guided vasculature may serve to further enhance vascularization of the myocardium 112 by promoting blood perfusion in the myocardium 112.

In the above embodiment, the formation of multiple microchannels using separately deployed microneedles that intersect one another's deployment paths are illustrated. However, embodiments in which separately deployed microneedles and/or sequentially formed microchannels using a single, or limited number of microneedles, are also contemplated.

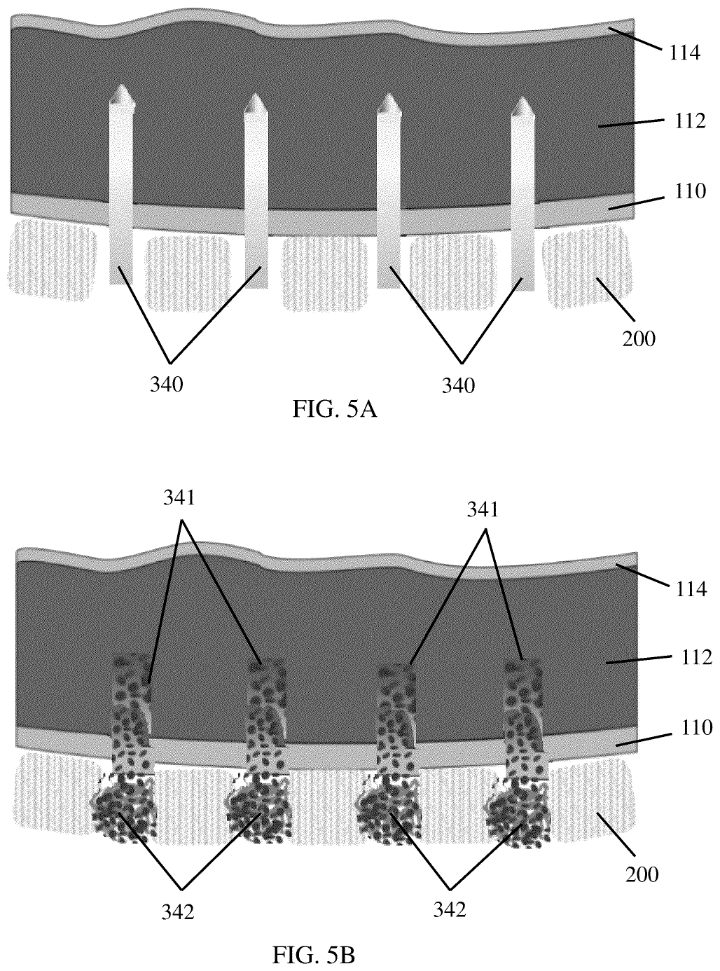

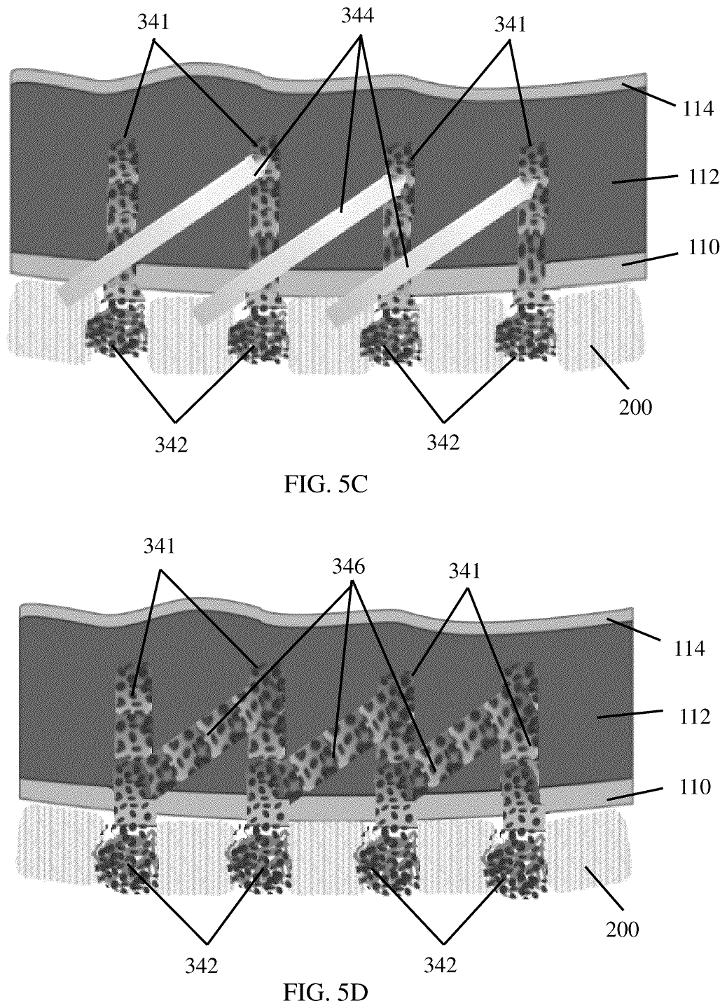

In another embodiment, guided interconnected vasculature may be made using a plurality of microneedles having a straight profile which may be inserted into a region of heart tissue in a first direction (e.g., substantially perpendicular to the heart tissue surface) to form a first plurality of microchannels. Another plurality of microneedles may then be inserted at an angle relative to the first plurality of microchannels to form a second plurality of microchannels, where at least a portion of the second plurality of microchannels may intersect two or more of the first plurality of microchannels to form a plurality of interconnected microchannels. FIG. 5A shows one such embodiment of a region of heart tissue having an applied EBM patch 200 where a plurality of straight microneedles 340 have been penetrated into the myocardium 112 of the heart of a patient in a first perpendicular orientation. Specifically, FIG. 5A shows the straight microneedles 340 being extended through the through holes formed in the EBM patch 200 and into the underlying heart tissue. As shown, the straight microneedles 340 extend through the epicardium 110 and partially into the myocardium 112 of the patient, but not through the endocardium 114 and into the ventricle. In FIG. 5A, the straight microneedles 340 are shown to be configured as coring needles, but any suitable needle type may be used such as pointed needles as the disclosure is not so limited.

FIG. 5B shows the embodiment of FIG. 5A after the straight microneedles 340 have been removed from the heart tissue of the patient, thereby forming microchannels 341 having blood flows 342. FIG. 5C shows the heart tissue of FIG. 5B with a second plurality of microneedles 344 inserted into the heart tissue at an angle relative (e.g., between 30 degrees and 60 degrees or other appropriate angle) to the existing first set of microchannels 341, with the second plurality of microneedles 344 being extended through the epicardium 110 and into the myocardium 112, but not through the endocardium 114. The second plurality of microneedles 344 are inserted such that at least a portion of the second plurality of microneedles 344 separately intersect with two or more of the first microchannels 341, thereby forming interconnected guided vasculature. FIG. 5D shows the embodiment of FIG. 5C after the second plurality of microneedles 344 have been removed from the heart tissue of the patient, thereby forming a second plurality of microchannels 346. At least a portion of the resulting second plurality of microchannels 346 separately intersect with two or more of the existing first plurality of microchannels 341, thereby forming intersecting guided vasculature in the heart tissue where a flow path may extend between multiple parallel microchannels. As disclosed herein, the formation of interconnected guided vasculature may serve to further enhance vascularization of the myocardium 112 by promoting blood perfusion in the myocardium 112.

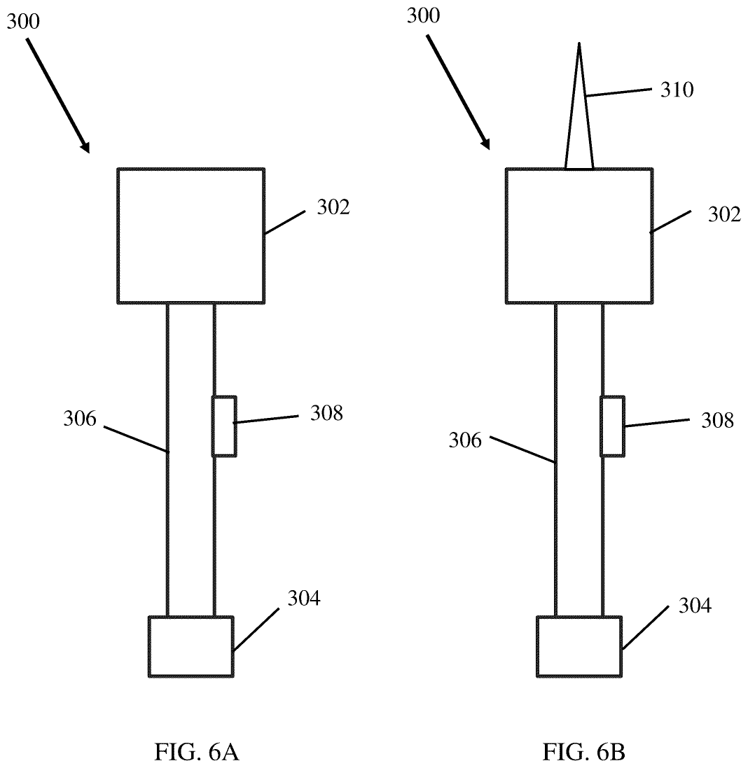

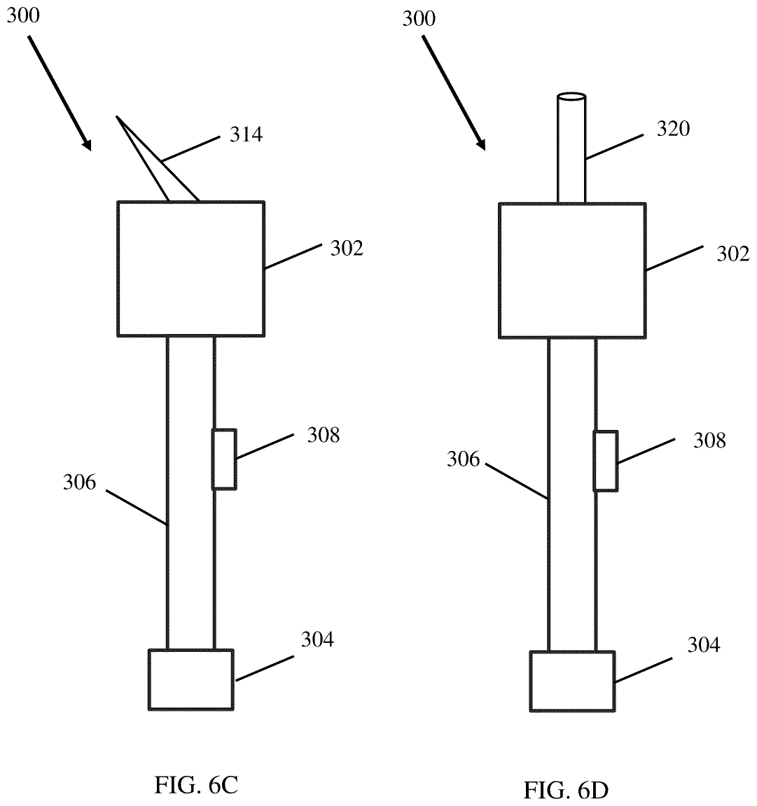

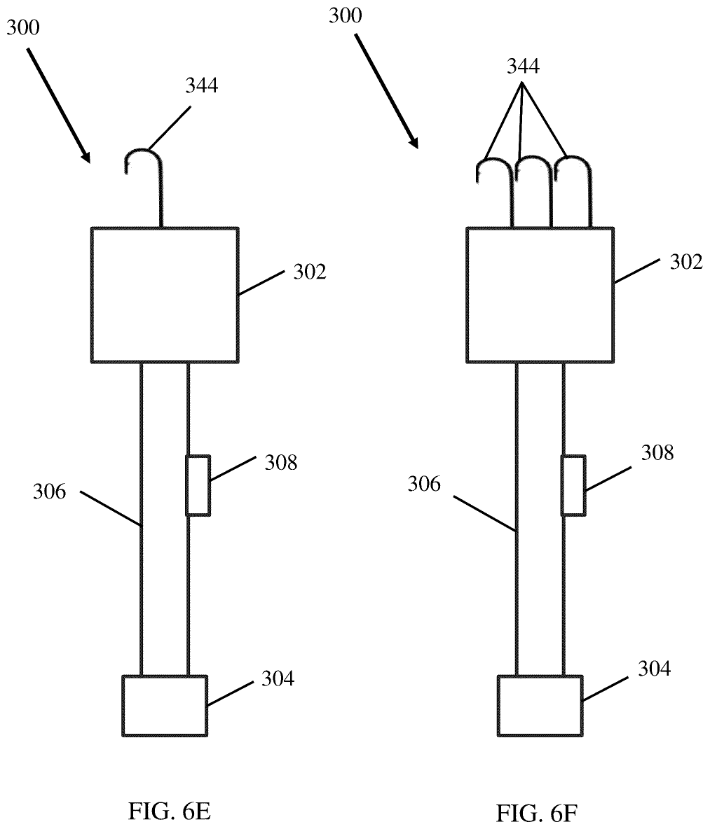

FIG. 6A shows an embodiment of a microneedling device 300 configured for use in penetrating a corresponding portion of heart tissue to form microchannels. The microneedling device 300 may include a distal portion 302, proximal portion 304, and a handle 306 disposed between these portions. The handle 306 may include a trigger 308 (e.g., a button, pull trigger, knob, or other appropriate type of trigger) configured to actuate a corresponding microneedle or plurality of microneedles between a retracted configuration and an extended configuration. While the trigger 308 is shown as being disposed on the handle 306 in the embodiment of FIG. 6A, the trigger 308 may be located on any portion of the microneedling device 300 or on a separate control mechanism as the disclosure is not so limited. In the embodiment of FIG. 6A, the microneedle or plurality of microneedles is not shown due to being in the retracted position in this embodiment. The microneedles may be of any suitable type, including pointed and/or coring microneedles. The microneedles may also be of any suitable profile, including straight, curved (e.g., hooked), angled, or any other suitable profile. Further, in some embodiments, the needles may also include coatings on the exterior needle surface that may serve to reduce needle friction when penetrating the heart tissue. In some embodiments, a suitable coating may include, but is not limited to water-based lubricants and silicone-based lubricants (e.g., silicone oil).

In some embodiments, any suitable actuator for actuating the microneedles between a retracted and an extended configuration in response to operation of a trigger may be used. Actuators may include, but are not limited to manual actuation arrangements (e.g., linear slides, gears and racks, etc.), spring loaded actuators, electrical solenoids, pneumatic actuators, and/or any other appropriate type of actuator capable of moving a microneedle between a retracted configuration within a housing of a device and an extended configuration extending at least partially out from the housing of the device with a desired penetration length. Additionally, an actuator may be used to actuate individual microneedles in a sequential manner, or may be used to actuate all of the microneedles simultaneously depending on the embodiment.

FIGS. 6B-6F show similar embodiments to that of FIG. 6A, but include a variety of one or more microneedles that are shown to be in the extended configuration, extending out of the distal portion 302 of the microneedling device 300. FIG. 6B shows an embodiment where the microneedle 310 is a pointed microneedle having a straight profile that extends out in a direction that is perpendicular to a surface of the distal portion of the device which may be configured to be placed in contact with heart tissue to be microneedled. By contrast, FIG. 6C shows an embodiment where the microneedle 314 is a pointed microneedle that is oriented such that a direction of extension of the microneedle may be angled relative to a surface the distal portion of the microneedling device is placed in contact with. In some embodiments, microneedles having an angled profile may be oriented at any suitable angle to permit penetration into the heart tissue of a patient. In some embodiments, it may be advantageous to incorporate a combination of needle types and profiles to create overlapping guided vascularization in the heart tissue. For example, similar to FIGS. 4A and 4B above, a plurality of straight microneedles may be employed to form a first plurality of microchannels, and then a plurality of angled microneedles may be employed to form a second plurality of microchannels that intersect the first plurality of microchannels, thereby forming overlapping guided vasculature.

FIG. 6D shows another embodiment of a microneedling device 300 where the one or more microneedles 320 is a coring microneedle with a straight profile that is extended in a direction that is perpendicular to a surface that the distal portion of the device is placed in contact with. As disclosed herein, the coring type microneedle may be used to remove portions of heart tissue approximately equal in size to the internal diameter of the coring microneedle.

FIG. 6E shows an embodiment of a microneedle 344 having a curved profile. In such an embodiment, the microneedle may be retracted into the microneedling device in a first configuration and as the microneedle is extended it may assume the depicted curved geometry. For example, super elastic alloys and/or polymers, including materials such nickel titanium alloys (e.g., Nitinol) an elastic material, may deformed from a first retracted configuration to the depicted curved shape in the extended configuration. Of course, rigidly deployed curved microneedles may also be used. FIG. 6F similarly shows an embodiment of microneedles 344 with a curved (e.g., hooked) profile. However, in the embodiment of FIG. 6F a plurality of microneedles 344 are shown being deployed into the extended configuration. While the embodiment of FIG. 6F shows a plurality of curved microneedles 344, any suitable type of microneedles disclosed herein may be employed for use in a microneedling device in any suitable number as the disclosure is not limited in this fashion.

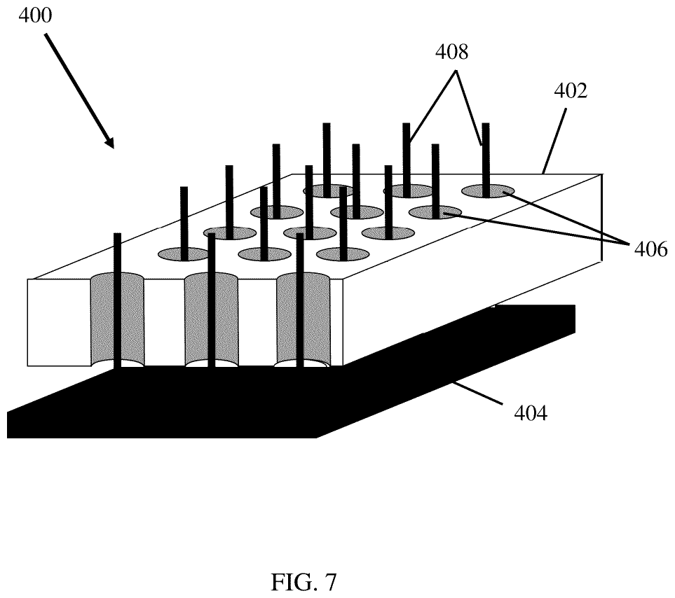

FIG. 7 shows an embodiment of a combined device 400 comprising a biopolymer scaffold material in the form of an EBM patch 402 and a microneedling device including a plurality of microneedles 408 attached to and supported on a supporting surface 404 of the microneedling device. The EBM patch 402 includes a plurality of through holes 406 disposed along the patch. The plurality of microneedles 408 may be aligned with and disposed in through holes of the EBM patch 402 such that the EBM patch may be disposed on the microneedling device. In some embodiments, the EBM patch 402 may not be pre-formed with through holes. In some such embodiments, the plurality of microneedles 408 may be configured to form through holes (e.g., slits, cutouts, etc) in the non-perforated EBM patch. When used, a corresponding region of heart tissue 404 may be penetrated by the plurality of microneedles 408 of the microneedling device to form microchannels in the heart tissue 404. The EBM patch, or other biopolymer scaffold material, may then be deposited onto the epicardium of the heart in a position and orientation where it the through holes of the EBM patch are aligned with the resulting microchannels formed in the heart.

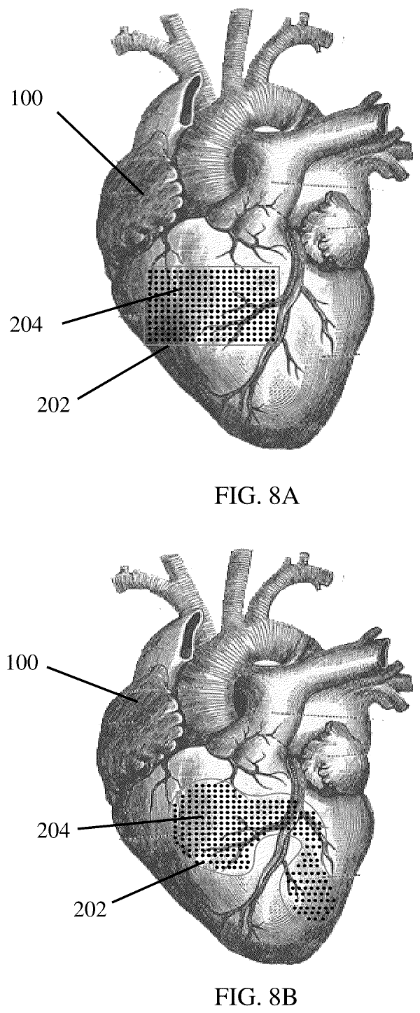

FIG. 8A shows an embodiment of a heart 100 with a biopolymer scaffold material in the form of an EBM patch 202 applied to the surface of the heart 100. The EBM patch 202 has a plurality of through holes in the form of through holes 204 as shown. The EBM patch 202 of FIG. 8A is shown to have a symmetrical rectangular shape. By contrast, FIG. 8B shows a heart 100 with an EBM patch 202 applied to the surface of the heart 100, with the EBM patch 202 having an asymmetrical shape. As discussed in reference to other embodiments herein, the EBM patch 202 may be customized to substantially match an ischemic region of heart tissue of a patient. Alternatively, or additionally, the asymmetric patch may be sized and shaped such that when applied to the heart, the patch may extend between healthy heart tissue and ischemic heart tissue which may help to guide angiogenesis and revascularization of the ischemic heart tissue. This is the case in FIG. 8B where an asymmetric patch extends between two different regions of the heart. Of course, any suitable patch shape and/or size may be employed depending on the condition to be treated as the disclosure is not so limited.

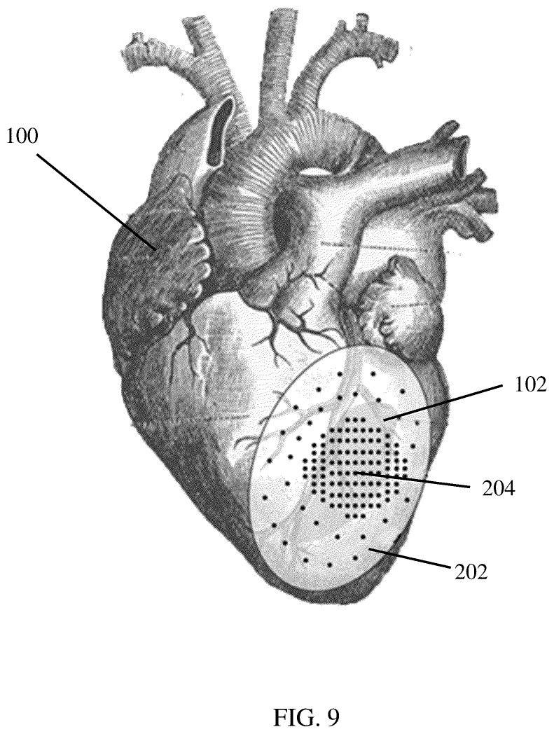

FIG. 9 shows an embodiment of a heart 100 with a biopolymer scaffold material in the form of an EBM patch 202 specifically applied to an ischemic region 102 of the heart 100 having a myocardial infarction. The EBM patch 202 further shows a plurality of through holes 204 formed in the patch with the plurality of through holes having a non-uniform distribution across the patch. As shown in FIG. 9, there may be a higher area density of through holes in a portion (e.g., an interior portion) of the patch aligned with the underlying ischemic region 102 of the heart. In contrast, the portion of the patch surrounding the ischemic region 102 (e.g., an outer border portion) of the patch may have a lower area density of through holes. The inventors have recognized that selectively forming through holes 204 on the EBM 202 as shown may serve to promote revascularization by directing angiogenesis from the surrounding non-ischemic regions of the heart to the ischemic region 102 while still offering strong mechanical properties of the patch in the outer border portion of the patch where the patch may be more securely attached to the underlying heart tissue.

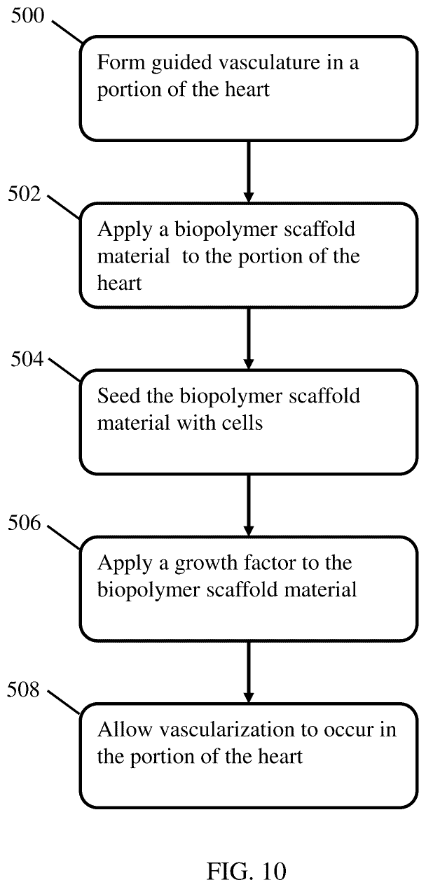

A method of forming vascularization in an ischemic portion of a heart is shown in FIG. 10. In step 500, guided vasculature may be formed in a portion of the heart. The portion of the heart may be an ischemic portion, and the guided vasculature may be formed by creating microchannels in the ischemic heart tissue. However, instances in which guided vasculature is formed in healthy heart tissue surrounding the ischemic portion of the heart are also contemplated.

In step 502, a biopolymer scaffold material may be applied to epicardium of the portion of the heart (e.g., the ischemic portion). As disclosed herein, the biopolymer scaffold material may take the form of an EBM patch, but other materials such as porcine may be used.

In step 504, the biopolymer scaffold material may be seeded with one or more populations of cells. The cells may be of any suitable type, including autologous cells or engineered allogenic cells. Alternatively, the biopolymer scaffold material may be pre-seeded with such cell populations prior to implantation.

Additionally, therapeutic compositions may also be included with the biopolymer scaffold material as disclosed elsewhere herein either prior to, or in some instances after, implantation. For example, in step 506, a growth factor may be applied to the biopolymer scaffold material. As noted herein, the growth factor may be used to promote revascularization and/or regeneration of the target heart tissue site (e.g., an ischemic portion).

In step 508, vascularization may be allowed to occur in the portion of the heart where guided vascularization is formed. While steps 504 and 506 are detailed in reference to FIG. 10, in some embodiments, vascularization may be caused through the use of guided vascularization (e.g., microchannels), the application of a biopolymer scaffold material including through holes to heart tissue, seeding of a biopolymer scaffold material disposed on heart tissue with one or more appropriate populations of cells, and/or the combination of the various therapies and devices disclosed herein.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. An ischemic heart repair device comprising:
a biopolymer scaffold material, wherein the biopolymer scaffold material includes a plurality of interconnected pores, wherein the biopolymer scaffold material has a thickness of equal to or greater than approximately 0.2 mm and less than or equal to approximately 6 mm;
a plurality of through holes formed in the biopolymer scaffold material, wherein the plurality of through holes extend from a first surface of the biopolymer scaffold material to a second opposing surface of the biopolymer scaffold material, wherein the plurality of through holes have an average maximum transverse dimension that is less than or equal to 1 mm.

2. The ischemic heart repair device of claim 1, wherein the biopolymer scaffold material comprises a decellularized extracellular matrix.

3. The ischemic heart repair device of claim 2, wherein the decellularized extracellular matrix is seeded with a population of at least one of autologous cells and engineered allogenic cells.

4. The ischemic heart repair device of claim 2, wherein the decellularized extracellular matrix comprises xenogeneic fetal or neonatal tissue.

5. The ischemic heart repair device of claim 2, wherein the decellularized extracellular matrix is decellularized bovine extracellular matrix or decellularized porcine extracellular matrix.

6. The ischemic heart repair device of claim 2, wherein the biopolymer scaffold comprises a therapeutic composition configured to be delivered to a heart of a subject.

7. (canceled)

8. The ischemic heart repair device of claim 1, wherein the biopolymer scaffold material is a patch.

9. The ischemic heart repair device of claim 1, wherein the biopolymer scaffold material has a porosity of greater than or equal to approximately 20% and less than or equal to approximately 70%.

10. The ischemic heart repair device of claim 1, wherein the biopolymer scaffold material has a tensile strength of greater than or equal to approximately 1 MPa and less than or equal to approximately 60 MPa.

11. The ischemic heart repair device of claim 1, wherein the biopolymer scaffold material has an area absorption capacity of liquid of greater than or equal to approximately 0.1 ml per square centimeter and less than or equal to approximately 1 ml per square centimeter.

12. The ischemic heart repair device of claim 1, wherein the biopolymer scaffold material has a suture retention strength of greater than or equal to approximately 5 N and less than or equal to approximately 500 N.

13. The ischemic heart repair device of claim 1, wherein the plurality of through holes include at least one selected from slits and cutouts.

14. A method of forming guided vascularization in heart tissue, the method comprising:
applying a biopolymer scaffold material having a plurality of interconnected pores to an epicardium of an ischemic portion of a heart of a subject, wherein the biopolymer scaffold material has a thickness of equal to or greater than approximately 0.2 mm and less than or equal to approximately 6 mm, and wherein a plurality of through holes is formed in the biopolymer scaffold material, wherein the plurality of through holes extend from a first surface of the biopolymer scaffold material to a second opposing surface of the biopolymer scaffold material, wherein the plurality of through holes have an average maximum transverse dimension that is less than or equal to 1 mm.

15. The method of claim 14, further comprising forming a plurality of microchannels in the ischemic portion of the subject's heart, wherein the plurality of microchannels extend through the epicardium and partially through a myocardium of the subject's heart.

16. The method of claim 14, further comprising forming a plurality of microchannels in the ischemic portion of the subject's heart, wherein the plurality of microchannels extend through an endocardium and into a ventricle of the subject's heart.

17. The method of claim 15, further comprising moving a plurality of microneedles between an extended configuration and a retracted configuration to form the plurality of microchannels.

18. The method of claim 15, further comprising moving a microneedle between an extended configuration and a retracted configuration a plurality of times to form the plurality of microchannels.

19. The method of claim 15, wherein forming the plurality of microchannels comprises using at least one of lasers, radiofrequency, and ultrasound to form the microchannels.

20. The method of claim 15, wherein the plurality of microchannels are substantially aligned with the plurality of through holes.

21. The method of claim 15, wherein the plurality of microchannels are not aligned with the plurality of through holes.

22. The method of claim 14, wherein an area density of the plurality of through holes is greater than or equal to 10% and less than or equal to 80% of an area of the biopolymer scaffold material.

23. The method of claim 14, wherein the biopolymer scaffold material comprises a decellularized extracellular matrix and a therapeutic composition.

24. The method of claim 23, further comprising seeding the decellularized extracellular matrix with a population of at least one of autologous cells and engineered allogenic cells.

25. The method of claim 23, wherein the decellularized extracellular matrix comprises xenogeneic fetal or neonatal tissue.

26. The method of claim 23, wherein the decellularized extracellular matrix is decellularized bovine extracellular matrix or decellularized porcine extracellular matrix.

27. (canceled)

28. The method of claim 23, further comprising introducing the therapeutic composition to the biopolymer scaffold material.

29. The method of claim 28, wherein introducing the therapeutic composition to the biopolymer scaffold material comprises introducing the therapeutic composition to the biopolymer scaffold material via soaking.

30. The method of claim 14, wherein the biopolymer scaffold material is a patch.

31. The method of claim 14, wherein the plurality of through holes include at least one selected from slits and cutouts.

32-81. (canceled)
